(19) United States
(12) Patent Application Publication
(10) Pub. No.: US 2024/0225779 A9
(43) Pub. Date: Jul. 11, 2024

(54) FLUID DEPLOYMENT MECHANISM FOR BIOPSY SITE MARKER

(71) Applicant: Devicor Medical Products, Inc., Cincinnati, OH (US)

(72) Inventor: Andrew P. NOCK, Dayton, OH (US)

(21) Appl. No.: 18/485,404

(22) Filed: Oct. 12, 2023

Related U.S. Application Data
(60) Provisional application No. 63/418,322, filed on Oct. 21, 2022.

(51) Int. Cl. A61B 90/00; A61B 10/02

(52) U.S. Cl. CPC A61B 90/39; A61B 10/0275; A61B 2090/3987

(57) ABSTRACT

A system for marking a biopsy site includes a biopsy site marker, a needle, and a fluid source. The biopsy site marker includes a non-absorbable marker element and a bioabsorbable carrier. The marker element is at least partially disposed within the carrier. The needle includes a marker lumen sized to receive the biopsy site marker. The fluid source is configured to communicate a pressurized fluid to the needle to advance the biopsy site marker through a portion of the needle and into the marker lumen.

### PRIORITY

This application claims priority to U.S. Provisional Application Ser. No. 63/418,322, entitled “Fluid Deployment Mechanism for Biopsy Site Marker,” filed on Oct. 21, 2022, the disclosure of which is incorporated by reference herein.

### BACKGROUND

A number of patients will have breast biopsies because of irregular mammograms and palpable abnormalities. Biopsies can include surgical excisional biopsies and stereotactic and ultrasound guided needle breast biopsies. In the case of image directed biopsy, the radiologist or other physician may take a small sample of the irregular tissue for laboratory analysis. If the biopsy proves to be malignant, additional surgery (e.g., a lumpectomy or a mastectomy) may be required. In the case of needle-based biopsies, the patient may return to the radiologist a day or more later, and the biopsy site (the site of the lesion) may need to be relocated in preparation for the surgery. An imaging system, such as ultrasound, magnetic resonance imaging (MRI) or x-ray may be used to locate the biopsy site. In order to assist the relocation of the biopsy site, a marker may be placed at the time of the biopsy.

The use of markers used after breast biopsies to mark the location where the biopsied tissue was removed is described in the following US patents: U.S. Pat. No. 6,083,524, entitled “Polymerizable biodegradable polymers including carbonate or dioxanone linkages,” issued Jul. 4, 2000; U.S. Pat. No. 6,162,241, entitled “Hemostatic tissue sealants,” issued Dec. 4, 2000; U.S. Pat. No. 6,270,464, entitled “Biopsy localization method and device,” issued Aug. 7, 2001; U.S. Pat. No. 6,356,782, entitled “Subcutaneous cavity marking device and method,” issued Mar. 12, 2002; U.S. Pat. No. 6,605,294, entitled “Methods of using in situ hydration of hydrogel articles for sealing or augmentation of tissue or vessels,” issued Aug. 12, 2003; U.S. Pat. No. 8,600,481, entitled “Subcutaneous cavity marking device,” issued Dec. 3, 2013 and U.S. Pat. No. 8,939,910, entitled “Method for enhancing ultrasound visibility of hyperechoic materials”, issued Jan. 27, 2015. All of these US patents are incorporated by reference in their entirety.

Examples of breast biopsy devices used in connection with marker deployment are described in US Pub. No. 2009/0216151, entitled “Biopsy Probe with Hypodermic Lumen,” published Aug. 27, 2009; and US Pub. No. 2021/0282754, entitled “Biopsy System with End Deploy Needle,” published Sep. 16, 2021, the disclosures of which are incorporated by reference herein in their entirety.

Placement of a marker at the biopsy site may be performed through the biopsy needle itself or other elements associated with the biopsy needle such as an introducer cannula, a targeting sheath, obturator, and/or etc. In some circumstances, the particular configuration of the device used for placement of the marker may present challenges with the placing the marker at the biopsy site reliably and accurately. For instance, when the biopsy needle is used for placement of the marker, the marker may be driven through a lateral aperture in the biopsy needle. This form of deployment of the marker may be desirable because the lateral aperture of the biopsy needle may also be used to collect biopsy samples, so its position may direct correspond to the biopsy site. However, there may be challenges associated with directing the marker axially through the biopsy needle and then laterally out of the lateral aperture. Accordingly, it may be desirable to use alternative features to deploy a marker to increase reliably and accuracy of placement of the marker.

While several systems and methods have been made and used for obtaining a biopsy sample and marking a biopsy site, it is believed that no one prior to the inventors has made or used the invention described in the appended claims.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements. In the drawings some components or portions of components are shown in phantom as depicted by broken lines.

FIG. 1 depicts a front elevational view of an example of a biopsy site marker;

FIG. 2 depicts a perspective view of an example of a marker delivery device;

FIG. 3 depicts a detailed perspective view of a marker introducer of the marker delivery device of FIG. 2;

FIG. 4 depicts a cross-sectional view of the marker introducer of FIG. 3 with the marker of FIG. 1 disposed within the marker introducer, the cross-section being taken along line 4-4 of FIG. 3;

FIG. 5 depicts a perspective view of an example of a biopsy device;

FIG. 6 depicts a cross-sectional view of a needle of the biopsy device of FIG. 5, the cross-section being taken along line 6-6 of FIG. 5;

FIG. 7A depicts detailed perspective view of a tissue sample holder of the biopsy device of FIG. 5, a plug being removed from an accessory port of the tissue sample holder;

FIG. 7B depicts another detailed perspective view of the tissue sample holder of FIG. 7A, the tissue sample holder being used in combination with the marker delivery device of FIG. 2;

FIG. 7C depicts yet another detailed perspective view of the tissue sample holder of FIG. 7A, the marker introducer of FIG. 3 being inserted into the accessory port of FIG. 7A;

FIG. 8A depicts a detailed cross-sectional view of the needle of FIG. 6, the needle being used in combination with the marker of FIG. 1;

FIG. 8B depicts another detailed cross-sectional view of the needle of FIG. 6, the marker of FIG. 1 being deployed through the needle;

FIG. 9 depicts a detailed cross-sectional view of an example of an alternative needle for use with the biopsy device of FIG. 5;

FIG. 10A depicts another detailed cross-sectional view of the needle of FIG. 9, the needle being used in combination with the marker of FIG. 1; and

FIG. 10B depicts yet another detailed cross-sectional view of the needle of FIG. 9, the marker of FIG. 1 being deployed from the needle.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

It may be beneficial to be able to mark the location or margins of a lesion, whether temporarily or permanently, prior to or immediately after removing or sampling it. Marking prior to removal may help to ensure that the entire lesion is excised, if desired. Alternatively, if the lesion were inadvertently removed in its entirety, marking the biopsy site immediately after the procedure would enable reestablishment of its location for future identification.

Once a marker is positioned at a biopsy site, it may be desirable for the marker to remain visible under ultrasound. It may also be desirable to make the marker readily identifiable relative to other structural features of a patient. For instance, it may be desirable for the marker to be distinguishable under ultrasound visualization from microcalcifications to avoid inadvertently characterizing the marker as a microcalcification during subsequent ultrasonic examinations. Generally, microcalcifications are used in the field to identify suspicious lesions or masses. Thus, it is generally desirable for the ultrasound view to be distinguishable as a marker and not inadvertently identified as a new mass.

### I. EXEMPLARY MARKER

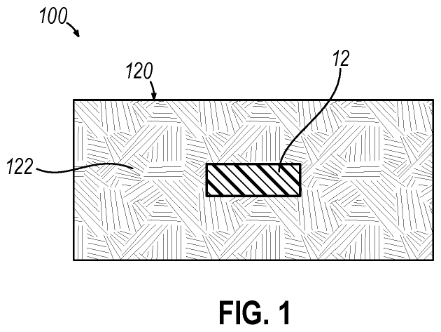

Aspects presented herein relate to devices and procedures for manufacturing a marker for percutaneously marking a biopsy cavity. For instance, as seen in FIG. 1, a marker (100) may be initially placed in the biopsy cavity to facilitate relocation of the biopsy site. Marker (100) may include a carrier (120) and a marker element (12). Carrier (120) generally includes a bioabsorbable marker material (122). Thus, carrier (120) is generally configured for absorption into a patient after placement of marker (100) within the biopsy cavity. In some versions, carrier (120) can include a plurality of microbubbles to enhance visualization of carrier (120) under ultrasound. As will be described in greater detail below, marker material (122) is generally bioabsorbable such that marker material (122) may be generally absorbed into the patient's tissue over time. In the present example, marker material (122) comprises a hydrogel that is initially in a dehydrated state. Although a hydrogel is used in the present example, it should be understood that in other examples marker material (122) may comprise other known bioabsorbable materials such as collagen, certain synthetic materials, or combinations of hydrogel with other materials.

In the present version, marker (100) further includes a marker element (12) that is generally not bioabsorbable (e.g., non-absorbable). Marker element (12) may comprise a radiopaque or echogenic marker embedded within the bioabsorbable marker material (122) of carrier (120). For instance, marker element (12) may include metal, hard plastic, or other radiopaque or hyperechoic materials known to those of ordinary skill in the art in view of the teachings herein. In other versions, marker (100) may be formed without a marker element (12). In still other versions, marker (100) may be formed with only marker element (12) such that carrier (120) is omitted and marker element (12) is in a “bare” form. In other words, in some examples, marker (100) is formed of only carrier (120) as a bare clip.

Marker material (122) is generally expandable once disposed within a patient at a biopsy site. In particular, the initially dehydrated marker material (122) may absorb fluid from the surrounding tissue into which it is inserted. In response to this absorption of fluid, marker material (122) may swell, thereby permitting carrier (120) to fill a cavity formed at a biopsy site by removal of tissue samples during a biopsy procedure. Biodegradable materials may be particularly suitable in applications where it is desired that natural tissue growth be permitted to completely or partially replace the implanted material over time. Accordingly, biocompatibility is ensured, and the natural mechanical parameters of the tissue are substantially restored to those of the pre-damaged condition.

Marker (100) may be inserted into the body either surgically via an opening in the body cavity, or through a minimally invasive procedure using such devices as a catheter, introducer or similar type insertion device. Marker (100) may be delivered immediately after removal of the tissue specimen using the same device used to remove the tissue specimen itself. Follow-up noninvasive detection techniques, such as x-ray mammography or ultrasound may then be used by the physician to identify, locate, and monitor the biopsy cavity site over a period of time via marker (100).

Marker (100) of the present version is large enough to be readily visible to a clinician under x-ray or ultrasonic viewing, for example; yet small enough to be able to be percutaneously deployed into the biopsy cavity and to not cause any difficulties with the patient. Although examples are described in connection with treatment and diagnosis of breast tissue, aspects presented herein may be used for markers in any internal, tissue, e.g., in breast tissue, lung tissue, prostate tissue, lymph gland tissue, etc.

The hydration of the marker material (122) of carrier (120) by the natural moisture of the tissue surrounding it causes expansion of the polymer and thus minimizes the risk of migration. The growing hydrogel-based marker material (122) centers marker (100) in the biopsy cavity as it grows. As the hydrogel expands, naturally present moisture from the surrounding tissue, the hydration enables increasing sound through transmission, appears more and more hypoechoic and is easy to visualize on follow-up ultrasound studies.

The hydrated hydrogel marker material (122) of carrier (120) may also be used to frame permanent marker element (12). The hypoechoic nature of the hydrated marker material (122) enables ultrasound visibility of the permanent marker element (12) within the hydrogel hydrated marker material (122) because the permanent marker element (12) is outlined as a specular reflector within a hypoechoic hydrated marker having a water-like nonreflective substrate.

### II. EXEMPLARY MARKER DELIVERY DEVICE WITH FLUID DEPLOYMENT

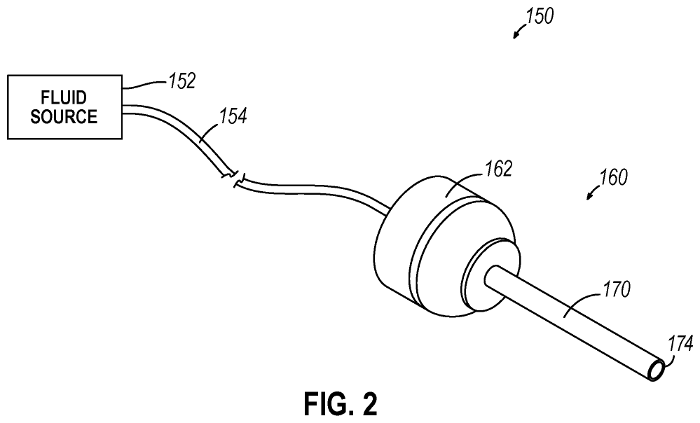

In some versions, it may be desirable to deploy marker (100) described above within the body cavity using certain marker delivery devices. For instance, FIGS. 2 through 4 show an exemplary marker delivery device (150) that is generally configured to deploy marker (100) at a biopsy site through various components associated with acquisition of biopsy samples such as a biopsy needle. Marker deliver device (150) of the present version includes a fluid source (152) and a marker introducer (160). Fluid source (152) in the present version is show schematically as fluid source (152) may include a variety of configurations. For instance, in some versions, fluid source (152) may include a cylindrical reservoir for containing a fluid and a plunger for driving the fluid from the cylindrical reservoir to marker introducer (160). In such versions, the plunger may be driven manually or by automatic means such as by an automatic fluid dispenser. In other versions, fluid source (152) may include a pressurized fluid reservoir and fluid may be released by actuating a valve disposed between fluid source (152) and marker introducer (160). In yet other versions, fluid source (152) may include a variety of other suitable configurations as will be appreciated by those of ordinary skill in the art in view of the teachings herein.

Fluid source (152) is generally configured to provide a pressurized fluid to marker introducer (160). As will be described in greater detail below, this pressurized fluid may then be used to drive marker (100) from marker introducer (160), through various elements associated with a biopsy procedure, and into position at a biopsy site. Thus, it should be understood that fluid source (152) may contain a variety of different fluids suitable for transport of marker (100). Generally, any suitable fluid may be used, provided the fluid is biocompatible as some fluid may incidentally be introduced into a patient. For instance, in some versions, fluid source (152) may contain a saline solution. In addition, or in the alternative, various additives may be added to the fluid. Examples of additives may include therapeutic agents, locating agents, fluorescent markers, and/or etc.

Fluid source (152) in the present version is coupled to marker introducer (160) using a flexible tube (154). Flexible tube (154) may be included in the present version to enhance the compatibility of marker deliver device (150) with a variety of biopsy device configurations. For instance, in some circumstances, a limited amount of space may be available for accessory components around a biopsy device. Thus, it may be desirable to place fluid source (152) in one location and place marker introducer (160) in another location. Of course, flexible tube (154) is merely optional and may be omitted in some versions. In versions where flexible tube (154) is omitted, fluid source (152) may be directly coupled to marker introducer (160).

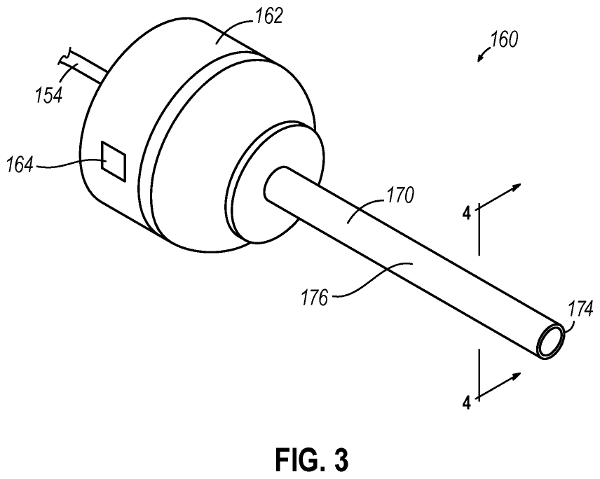

FIG. 3 shows marker introducer (160) in greater detail. As can be seen, marker introducer includes a hub (162) and a cannula (170) extending distally from hub (162). Hub (162) in the present version is coupled at a proximal end to flexible tube (144). In other versions, hub (162) may coupled to flexible tube (144) at a variety of alternative positions. For instance, in some versions, hub (162) may include a laterally extending port that may be used for coupling of flexible tube (144) at an angle relative to the longitudinal axis defined by cannula (170).

Hub (162) in the present version is generally configured to enhance manipulation of marker introducer (160). However, in some versions, hub (162) may be omitted and marker introducer (160) may alternatively be manipulated directly by cannula (170). In such versions, cannula (170) may couple directly to fluid source (152) either by flexible tube (144) or by a direct coupling with fluid source (152).

As described above, cannula (170) extends distally from hub (162). As will be described in greater detail below, cannula (170) is generally configured to engage a portion of a biopsy device or another component associated with a biopsy procedure to communicate marker (100) into the biopsy device or other component for deployment of marker (100) into tissue. Cannula (170) includes a generally tubular structure defining a hollow interior (172) and open distal end (174).

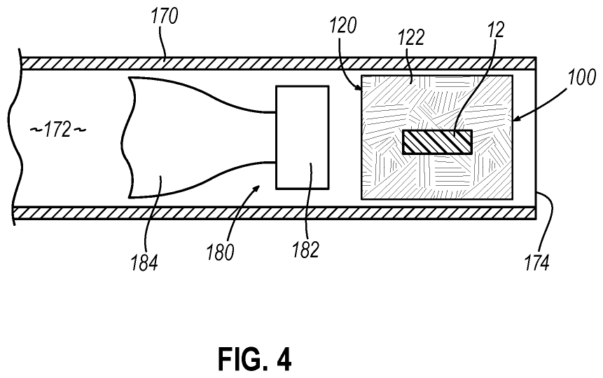

As best seen in FIG. 4, hollow interior (172) of cannula (170) is generally configured to receive marker (100) therein. Although not shown, it should be understood that the interior of cannula (170) may define one or more features configured to releasably hold marker (100) proximally of open distal end (174) until deployment of marker (100) is desired. Suitable features configured to releasably hold marker (100) may include, for example, one or more detents, one or more ribs, and/or etc. In other versions, marker (100) may be held in position within hollow interior (172) of cannula (170) by an interference fit between the inner diameter of cannula (170) and the outer diameter of marker (100).

In some versions, hollow interior (172) of cannula (170) may be equipped with an optional marker plug (180) as shown in FIG. 4. Marker plug (180) may be generally configured to promote movement of marker (100) through cannula (170) and through components of a biopsy device or other components associated with a biopsy procedure.

To facilitate such function, marker plug (180) may include a drive portion (182) and a sealing portion (184). Drive portion (182) may be generally configured to abut marker (100) and drive marker (100) through cannula (170) and other components used in connection with cannula (170), as will be described in greater detail below. Thus, drive portion (182) may include a generally rigid material configured to apply a force to marker (100).

Sealing portion (184) may extend proximally from drive portion (182) and may be configured to sealingly engage hollow interior (172) of cannula (170). In particular, sealing portion (184) may include a generally resilient yet pliable material configured to expand outwardly against the interior of cannula (170). Thus, sealing portion (184) may define a generally cup, flared, and/or conical shaped configuration. As will be described in greater detail below, the proximal face of sealing portion (184) may be configured to catch fluid communicated from fluid source (152). This action may then be used to simultaneously drive sealing portion (184) outwardly into engagement with the interior diameter of cannula (170) (or other similar components) while driving marker plug (180) distally.

Returning to FIG. 3, marker introducer (160) may include a variety of sensors (164, 176) configured to provide operational feedback to an operator. For instance, hub (162) may include a sensor (164) configured to sense fluid pressure being communicated from fluid source (152). Such fluid detection may be desirable in some circumstances to prevent the application of fluid beyond a predetermined pressure threshold, thereby preventing movement of marker (100) with excessive force.

In addition, or in the alternative, cannula (170) may include a sensor (176) configured to detect the presence of marker (100). Such detection of marker (100) may be desirable to provide feedback to an operator indicating when marker (100) exits cannula (170). In some versions, detection of marker (100) may be based on fluid pressure. For instance, a fluid pressure change of one predetermined amount may indicate marker (100) exiting cannula (100), while a fluid pressure change of another predetermined amount may indicate deployment of marker (100) within tissue. Thus, sensor (176) may be configured to provide feedback for a variety of operational conditions.

Although sensors (164, 176) are used in the present version to provide the feedback as described herein, it should be understood that in other versions, any one or more sensors (164, 176) may be replaced with other components suitable to provide operational feedback. For instance, in applications where sensors (164, 176) are used to prevent excessive fluid pressure, such sensors (164, 176) may be replaced with pressure release valves, release mechanisms, and/or mechanical indicators to provide tactile and/or visual feedback. Similarly, where sensors (164, 176) are used to detect certain operational conditions such as the progression of marker (100) relative to cannula (170), one or more of such sensors (164, 176) may be replaced with mechanical indicators used to provide tactile and/or visual feedback. Other suitable alternatives to sensors (164, 176) will be apparent to those of ordinary skill in the art in view of the teachings herein.

Although the present version includes a sensor (164, 176) configuration with one sensor (164) associated with hub (162) and another sensor (176) associated with cannula (170), it should be understood that in other versions various alternative sensor (164, 176) configurations may be used. For instance, in some versions, sensors (164, 176) may be consolidated within hub (162) or cannula (170). In addition, or in the alternative, although only two sensors (164, 176) are used in the present version, in other versions, a plurality of sensors (164, 176) may be used including sensor arrays having multiple sensors (164, 176) with multiple functions at a single location. Of course, various alternative sensor (164, 176) configurations will be apparent to those of ordinary skill in the art in view of the teachings herein.

### III. EXAMPLE BIOPSY DEVICE FOR USE WITH FLUID DEPLOYED MARKER

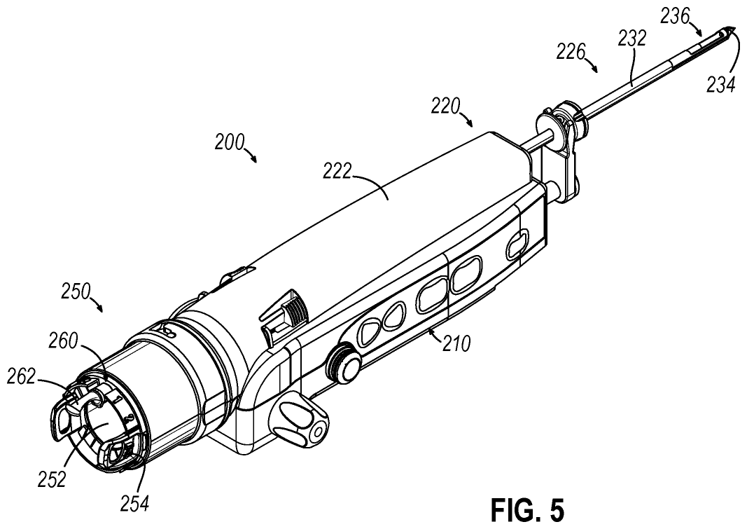

FIG. 5 shows an example of a biopsy device (200) for use with marker (100) and marker delivery device (150) described above. Biopsy device (200) includes a holster (210), a probe (220) and a tissue sample holder (250) extending proximally from a portion of probe (220). Probe (220) is separable from holster (210). By way of example only, probe (220) may be provided as a disposable component, while holster (210) may be provided as a reusable component. Use of the term “holster” herein should not be read as requiring any portion of probe (220) to be inserted into any portion of holster (210). Indeed, in some versions of biopsy device (200), probe (220) may simply sit on holster (210). In some other versions, a portion of holster (210) may be inserted into probe (220). Furthermore, in some versions of biopsy device (200), probe (220) and holster (210) may be of unitary or integral construction, such that the two components cannot be separated or are not identifiable as different components. Still other suitable structural and functional relationships between probe (220) and holster (210) will be apparent to those of ordinary skill in the art in view of the teachings herein.

Holster (210) of the present version is generally configured to drive various functions of probe (220). To facilitate such functions, holster (210) may thus include a needle rotation mechanism (not shown), a needle firing mechanism (not shown), a cutter drive mechanism (not shown), and a tissue holder rotation mechanism (not shown). The needle rotation mechanism may be operable to rotate a needle (226) about its longitudinal axis. The needle firing mechanism may be operable to fire needle (226) into tissue. The cutter drive mechanism may be operable to cause a cutter (246) to rotate and translate; while the tissue holder rotation mechanism may be operable to cause at least a portion of tissue sample holder (250) to rotate. Suitable components and structures that may be used to provide any of these mechanisms, as well as other optional features of holster (210), are disclosed in U.S. Non-Provisional patent application Ser. No. 11/942,764, filed Nov. 20, 2007, and entitled “Vacuum Timing Algorithm for Biopsy Device,” the disclosure of which is incorporated by reference herein. Of course, any other suitable components, structures, or configurations may be used. Alternatively, any such mechanisms may simply be omitted altogether.

Probe (220) includes a body (222) and a needle (226) extending distally from body (222). Tissue sample holder (250) may be removably secured to body (222), though tissue sample holder (250) may alternatively be secured to some other component. Although not shown, it should be understood that one or more tubes may be coupled with probe (220) for communication of fluids (e.g., vacuum, saline, atmospheric air, pressurized air, etc.) to needle (226) and/or tissue sample holder (250).

Needle (226) includes an outer cannula (232) having a tissue piercing tip (234) and a transverse tissue receiving aperture (236) (also referred to as a lateral aperture). The interior of outer cannula (232) of the present example defines a cannula lumen (238) (also referred to as a cutter lumen or axial lumen) and a vacuum lumen (240) (also referred to as a lower lumen or lateral lumen), with a wall (242) separating cannula lumen (238) from vacuum lumen (240). A plurality of fluid openings (244) may be formed in wall (242) to provide fluid communication between cannula lumen (238) and vacuum lumen (240). Of course, as with other components described herein, such openings are merely optional.

A hollow tubular cutter (246) may be slidably disposed within cannula lumen (238) of needle (226). Cutter (246) is generally configured to rotate and translate relative to needle (226) to sever one or more tissue samples from tissue prolapsed into aperture (236) of needle (226). Thus, cutter (246) may translate relative to aperture (236) to open and close aperture (236) either to sever tissue samples or to faciliate one or more other functions during a biopsy procedure. As described above, cutter (246) may be driven by one or more components of holster (210). For instance, in some versions, a cutter drive (not shown) may be partially disposed within body (222). Such a cutter drive may then be driven by one or more gears extending from a portion of holster (210).

Tissue sample holder (250) is configured to receive one or more tissue samples severed by cutter (246) and transported through a hollow interior of cutter (246). It should be understood that tissue sample holder (250) may take on a variety of forms and is not limited by the particular configuration shown and described herein. In the present version, tissue sample holder (250) includes a rotatable member (252) that is configured to receive one or more sample trays (254). Although not shown, it should be understood that each sample tray (254) may include a plurality of strips. Each strip may be configured to receive one or more tissue samples as rotatable member (252) is rotated to successively fill each strip with one or more severed tissue samples.

Rotatable member (252) further includes an access port (260) configured to removably receive an access plug (262). As will be described in greater detail below, access port (260) is configured to selectively provide access to the hollow interior of cutter (246) depending on the rotational position of rotatable member (252). Access port (260) in the present version is disposed between two sample trays (254). In this position, rotatable member (252) may be rotated to align access port (260) with a proximal end of cutter (246) rather than sample trays (254). Once aligned, access port (260) may be in communication with the hollow interior of cutter (246). Access port (260) may additionally provide a substantially linear path through tissue sample holder (250) and into cutter (246). In other versions, access port (260) may be positioned separately from sample trays (254) to promote access to the hollow interior of cutter (246) while still permitting sample collection using sample trays (254).

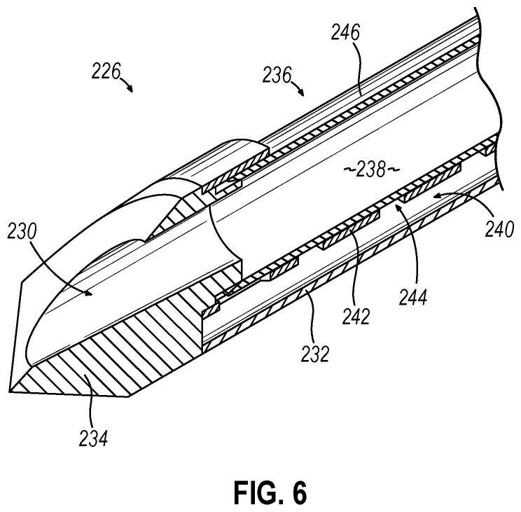

As best seen in FIG. 6, needle (226) further includes a marking lumen (230) extending through tissue piercing tip (234). As will be described in greater detail below, marking lumen (230) is generally configured to permit deployment of marker (100) through tissue piercing tip (234). Marker lumen (230) is positioned within tissue piercing tip (234) to communicate with cannula lumen (238). Marker lumen (230) is further positioned within tissue piercing tip (234) to align with a generally planar surface of tissue piercing tip (234) rather than intersecting with one or more cutting edges. In this configuration, isolation of marker lumen (230) to a planar surface of tissue piercing tip (234) may be desirable to preserve the tissue piercing function of tissue piercing tip (234) while providing access to cannula lumen (238).

Although marking lumen (230) of the present version is configured to communicate with cannula lumen (238), it should be understood that marker lumen (230) may be in communication with vacuum lumen (240) in other versions. In still other versions, marking lumen (230) may be configured to communicate with an entirely separate lumen extending through needle (226). For instance, in such versions, needle (226) may include an additional tubular structure adjacent to portions of wall (242) and/or outer cannula (232). Such a tubular structure may be configured to facilitate the transport of marker (100) through needle (226) to making lumen (230).

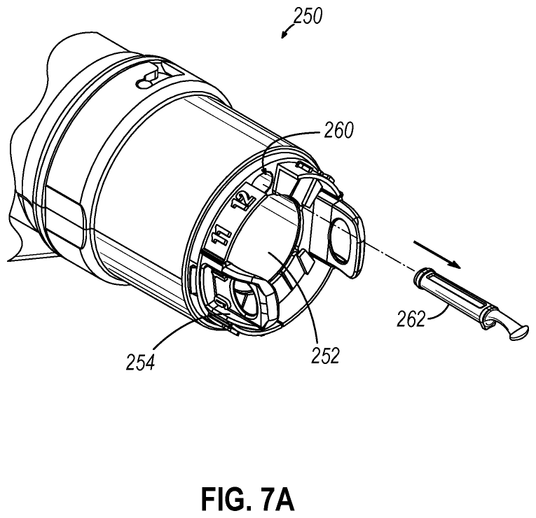

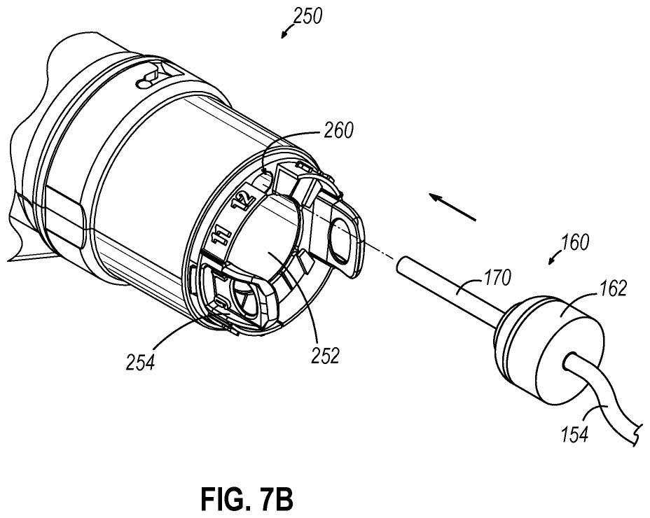

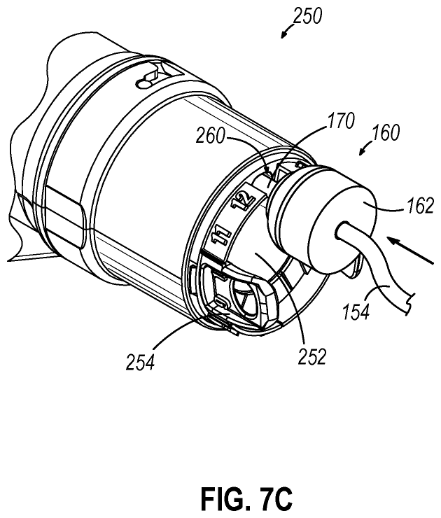

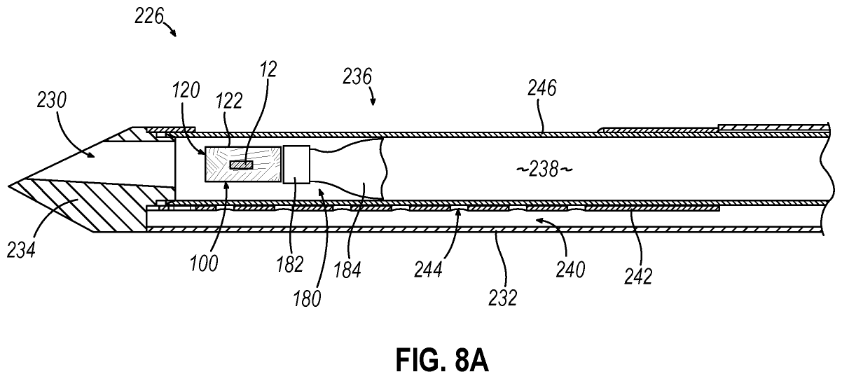

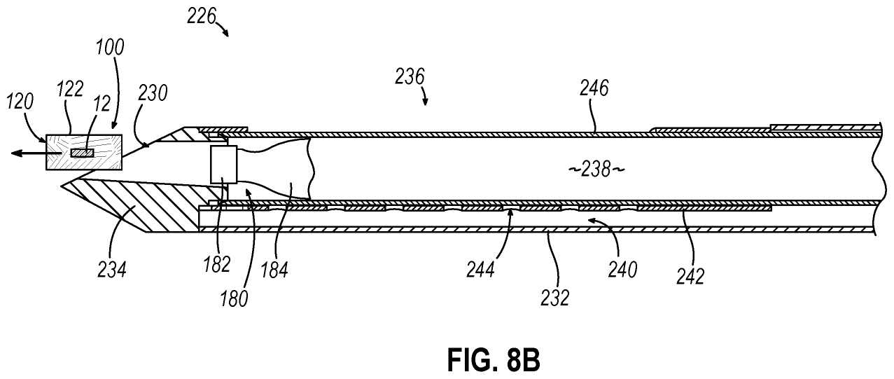

FIGS. 7A through 8B show an example of using biopsy device (200) in combination with marker delivery device (150) to deploy marker (100) at a biopsy site. In the present use, cutter (246) of probe (220) is generally used to guide marker (100) from marker delivery device (150) into marking lumen (230) of tissue piercing tip (234). Thus, prior to the use shown, biopsy device (200) may be used to collect one or more tissue samples from a region of tissue. Once the one or more tissue samples are collected, biopsy device (200) may remain in position within the region of tissue to perform the marking procedure described herein.

As best seen in FIG. 7A, biopsy device (200) may initially be prepared for marking. In particular, tissue sample holder (250) may be manipulated to move access port (260) rotatable member (252) into alignment with cutter (246). In the present configuration, this alignment is achieved by rotating rotatable member (252) to position access port (260) in the twelve o'clock position. Once alignment is performed, access plug (262) may be removed as shown in FIG. 7A to prepare alignment port (260) for use with marker delivery device (150).

After biopsy device (200) is prepared for marking, marker delivery device (150) may be placed into communication with cutter (246). In particular, cannula (170) of marker delivery device (150) may be inserted into access port (260) of tissue sample holder (250) as shown in FIGS. 7B and 7C. At this stage, the distal end of cannula (170) may be in fluid communication with cutter (246) such that marker delivery device (150) may be operable to communicate fluid into cutter (246) towards marking lumen (230) of tissue piercing tip (234).

Once cannula (170) of marker delivery device (150) is inserted into access port (260) of tissue sample holder (250), marker delivery device (150) may be used to deploy marker (100) through cutter (246). In particular, fluid source (152) may be actuated to communicate a pressurized fluid from fluid source (152) and intro cannula (170). The pressurized fluid may then drive marker (100) and marker plug (180) distally though cannula (170) and into cutter (246). As best seen in FIG. 8A, the presence of pressurized fluid may expand sealing portion (184) of marker plug (180) to engage the interior wall of cutter (246) to facilitate distal movement of marker plug (180) and marker (100).

Once marker (100) travels through cutter (246) marker (100) may be directed into marking lumen (230) of tissue piercing tip (234) as best seen in FIG. 8B. In particular, cutter (246) may be advanced within needle (226) to close aperture (236) and provide a generally linear path from cutter (246) and into marking lumen (230). At least a portion of marker plug (180) may also be driven into marking lumen (230) to drive marker (100) distally out of marking lumen (230) and out of tissue piercing tip (234). However, in some uses, at least a portion of sealing portion (184) may engage a portion of marking lumen (230) to hold marker plug (180) within marking lumen (230). In such uses, holding marker plug (180) may be desirable to maintain the pressurized fluid within needle (226). In other uses, marker plug (180) may be omitted entirely and pressurized fluid may also enter tissue along with marker (100). In such uses, pressurized fluid may optionally include therapeutic agents, marking agents, and/or etc.

Once marker (100) is deployed within tissue, biopsy device (200) may be extracted from the tissue. Marker (100) may then remain in position and may be used in subsequent follow-up procedures to identify the location of the biopsy site under various imaging modalities. Suitable imaging modalities may include, for example, ultrasound, x-ray, MRI, and/or etc.

During use of marker delivery device (150) for marking, sensors (164, 176) may be used for a variety of operational control features. For instance, one or more of sensors (164, 176) may be used throughout the procedure to detect the operational pressure of the pressurized fluid from fluid source (152). In some uses, the operational pressure may be directly communicated to an operator by use of a visual indicator, gauge, digital readout, and/or etc. In addition, or in the alternative, the operational pressure may be used to cutoff fluid source (152) or otherwise lock fluid source (152) upon detection of an operational pressure exceeding a predetermined threshold.

One or more sensors (164, 176) may also be used throughout the marking procedure to identify the position of marker (100) relative to one or more portions of marker delivery device (150). For instance, one or more sensors (164, 176) may be used to identify one or more changes in operational pressure. Such changes in operational pressure may be correlated to certain actions with marker (100). For instance, a certain change in pressure might occur when marker (100) transitions from cannula (170) to cutter (246), from cutter (246) to marking lumen (230), or from marking lumen (230) to tissue. Alternatively, one or more sensors (164, 176) may be configured to act as a proximity sensor to detect the distance between the proximity sensor and one or more portions of marker (100). Thus, one or more sensors (164, 176) may be used to communicate the position of marker (100) to an operator during the marking procedure.

### IV. EXAMPLE OF ALTERNATIVE NEEDLE FOR USE WITH BIOPSY DEVICE

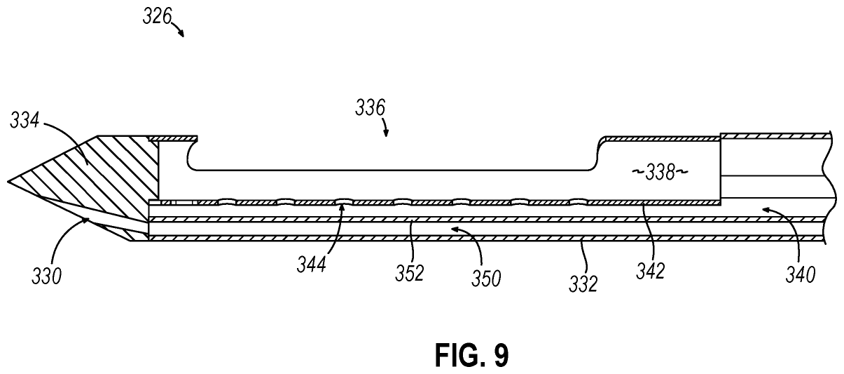

FIG. 9 shows an example of an alternative needle (326) that may be incorporated into biopsy device (200) described above in-lieu of needle (226). Needle (326) of the present version is substantially similar to needle (226) described above, except where otherwise described herein. For instance, like with needle (226) described above, needle (326) of the present version includes an outer cannula (332) having a tissue piercing tip (334) and a transverse tissue receiving aperture (336) (also referred to as a lateral aperture). As with outer cannula (232) described above, the interior of outer cannula (332) of the present version defines a cannula lumen (338) (also referred to as a cutter lumen or axial lumen) and a vacuum lumen (340) (also referred to as a lower lumen or lateral lumen), with a wall (342) separating cannula lumen (338) from vacuum lumen (340). A plurality of fluid openings (344) may be formed in wall (342) to provide fluid communication between cannula lumen (338) and vacuum lumen (340). Of course, as with other components described herein, such openings are merely optional.

Unlike needle (226) described above, needle (326) of the present version includes a dedicated marker transport lumen (350) for deployment of marker (100) rather than through cutter (246). As can be seen, marker transport lumen (350) extends axially the length of needle (326) proximate and parallel to cannula lumen (338) and vacuum lumen (340). Although marker transport lumen (350) is adjacent to vacuum lumen (340) in the present version, it should be understood that in other versions, marker transport lumen (350) may be defined in a variety of positions about outer cannula (332). For instance, marker transport lumen (350) may be oriented to one side in some versions. In such versions, marker transport lumen (350) may be either adjacent to cannula lumen (338), vacuum lumen (340), or both.

Marker transport lumen (350) is defined by an inner wall (352) in the present version. Inner wall (352) may be formed by an integral portion of outer cannula (332) in some versions, or as a separate component in other versions. In the present version, inner wall (352) is configured to fluidly isolate marker transport lumen (350) from other portions of needle (326) such as cannula lumen (328) and vacuum lumen (340). Such fluid isolation may be desirable in some versions to promote isolation of fluid from marker delivery device (150) from other portions of needle (326) and to contain pressure associated with the fluid. In other versions, one or more portions of inner wall (352) may be broken, open, or otherwise in communication with other portions of needle (326). Such a configuration of inner wall (352) may be desirable in some versions to promote ease of assembly or to reduce materials used for needle (326). In yet other versions, inner wall (352) may be omitted entirely and marker transport lumen (350) and vacuum lumen (340) may overlap with each other.

Although not shown, it should be understood that the proximal end of marker transport lumen (350) may be configured to communicate either directly with marker delivery device (150) or indirectly through tissue sample holder (250) or a port proximate tissue sample holder (250). For instance, in some versions, one or more portions of marker delivery device (150) may be incorporated into probe (220) or holster (210) to coupled directly to marker transport lumen (350). In other versions, tissue sample holder (250) may be configured to communicate with marker transport lumen (350) via access port (260) similarly to communication with cutter (246), but offset (e.g., six o'clock position rather than twelve o'clock position). In yet other versions, marker transport lumen (350) may be coupled to a tube (not shown), which may extend proximally from a portion of probe (220). Such a tube may be configured to receive cannula (170) of marker delivery device (150) for communication of marker (100) to marker transport lumen (350).

The distal end of marker transport lumen (350) is in communication with a marking lumen (330) extending through tissue piercing tip (334). Marking lumen (330) is similar to marking lumen (230) described above in that marking lumen (330) is sized to receive marker (100) so that marker (100) may be deployed through tissue piercing tip (334). Similarly, marking lumen (330) may be positioned relative to tissue piercing tip (334) so as to intersect with only a planar surface and not any cutting edge. As described above, such a configuration may be desirable to promote the tissue piercing function of tissue piercing tip (334), while also permitting deployment of marker (100) through tissue piercing tip (334).

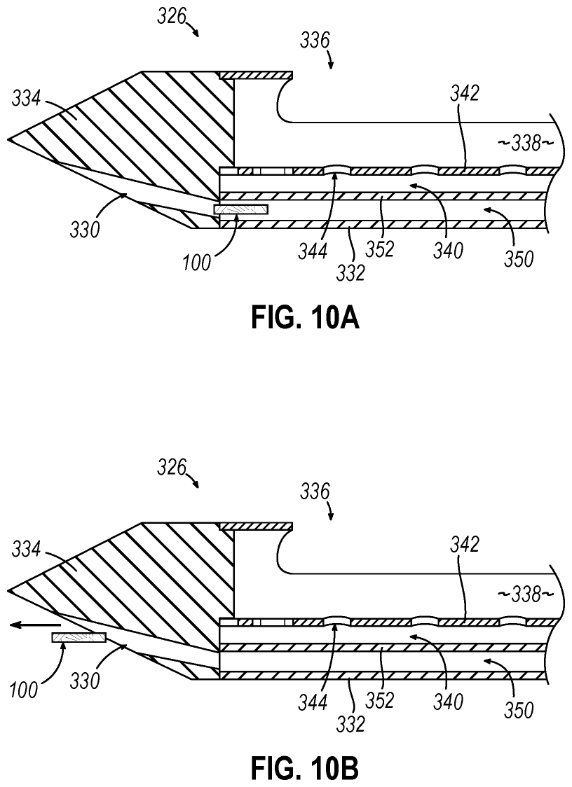

FIGS. 10A through 10B show an example of a use of needle (326) for deployment of marker (100) within tissue using marker deliver device (150). The use with respect to needle (326) of the present version is substantially similar to the use described above with respect to needle (226). For instance, as described above, biopsy device (200) may initially be prepared for marking. Such initial preparations may include, for example, collecting one or more tissue samples, aligning tissue sample holder (250) to communicate with marker transport lumen (350), and coupling cannula (170) of marker delivery device (150) to biopsy device (200) to communicate with marker transport lumen (350).

Once such initial preparations are completed, fluid source (152) of marker delivery device (150) may be actuated to release a pressurized fluid into cannula (170). The pressurized fluid may then act on marker (100) to drive marker (100) out of cannula (170) and into marker transport lumen (350). Marker (100) may then move distally though marker transport lumen (350) as shown in FIG. 10A. Once marker (100) reaches the distal end of marker transport lumen (350), marker (100) may then progress into marking lumen (330) and out of tissue piercing tip (334) as shown in FIG. 10B.

Although not shown, it should be understood that in some uses with needle (326) a marker plug similar to marker plug (180) described above may also be used. In such uses, the marker plug may similarly engage marking lumen (330) to be held within marking lumen (330), thereby preventing the pressurized fluid from exiting marking lumen (330). In other uses, structures similar to marker plug (180) may be omitted entirely as shown in FIGS. 10A and 10B. In such uses, the pressurized fluid may also exit tissue piercing tip (334), where the pressurized fluid may be used for enhancement of marking and/or for therapeutic purposes.

### V. EXEMPLARY COMBINATIONS

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

A system for marking a biopsy site, the system comprising: a biopsy site marker, the biopsy site marker including a non-absorbable marker element and a bioabsorbable carrier, the marker element being at least partially disposed within the carrier; a needle, the needle including a marker lumen sized to receive the biopsy site marker; a fluid source, the fluid source being configured to communicate a pressurized fluid to the needle to advance the biopsy site marker through a portion of the needle and into the marker lumen.

### Example 2

The system of Example 1, the needle including a tissue piercing tip, the marker lumen extending through a portion of the tissue piercing tip.

### Example 3

The system of Example 1, further comprising a cutter, the needle including a tissue piercing tip, an outer cannula, and a lateral aperture, the cutter being configured to move relative to the lateral aperture to sever one or more tissue samples, the marker lumen extending through a portion of the tissue piercing tip, the marker lumen being in communication with the cutter.

### Example 4

The system of Example 1, further comprising a cutter, the needle including a tissue piercing tip, an outer cannula, and a lateral aperture, the outer cannula defining a cutter lumen and a lateral lumen, the cutter being configured to move relative to the lateral aperture within the cutter lumen to sever one or more tissue samples, the marker lumen extending through a portion of the tissue piercing tip, the marker lumen being in communication with a portion of the lateral lumen.

### Example 5

The system of Example 1, further comprising a cutter, the needle including a tissue piercing tip, an outer cannula, and a lateral aperture, the outer cannula defining a cutter lumen, a lateral lumen, and a marker transport lumen, the cutter being configured to move relative to the lateral aperture within the cutter lumen to sever one or more tissue samples, the marker lumen extending through a portion of the tissue piercing tip, the marker lumen being in communication with a portion of the marker transport lumen.

### Example 6

The system of Example 5, the marker transport lumen being fluidly isolated from the cutter lumen and the lateral lumen.

### Example 7

The system of any of Examples 5 or 6, the marker transport lumen being disposed adjacent to the lateral lumen.

### Example 8

The system of any of Examples 1 through 7, further comprising a marker delivery device, the fluid source being in communication with the marker delivery device, the marker delivery device being configured to releasably receive the biopsy site marker.

### Example 9

The system of any of Examples 1 through 7, further comprising a maker delivery device, the fluid source being in communication with the marker delivery device, the marker delivery device including a cannula and a hub, the cannula being configured to receive the biopsy site marker, the cannula being further configured to communicate with the needle to transport the biopsy site marker from a portion of the cannula to the needle.

### Example 10

The system of any of Examples 8 or 9, the marker delivery device including a sensor, the sensor being configured to detect a fluid pressure within a portion of the marker delivery device.

### Example 11

The system of any of Examples 8 through 10, the marker delivery device including a release mechanism in communication with an interior of the marker delivery device, the release mechanism being configured to release fluid in response to contact with a fluid pressure above a predetermined threshold.

### Example 12

The system of any of Examples 1 through 11, further comprising a tissue sample holder, the tissue sample holder being in communication with a portion of the needle, the tissue sample holder having an access port configured to permit selective communication between the fluid source and the needle via the tissue sample holder.

### Example 13

The system of Example 12, the access port of the tissue sample holder being configured to provide a substantially linear path through the tissue sample holder to the needle.

### Example 14

The system of any of Examples 1 through 11, further comprising an access tube, the access tube being in communication with one or more portions of the needle, the access tube being configured to permit communication of fluid from the fluid source to the needle.

### Example 15

The system of any of Examples 1 through 11, further comprising an access port, the access port being configured to provide direct access to an interior of the needle for fluid communication from the fluid source.

### Example 16

An apparatus for collecting a biopsy sample and marking tissue, the apparatus comprising: a body; a needle extending distally from the body, the needle including a tissue piercing tip, an outer cannula, and a marking lumen extending through the tissue piercing tip and in communication with an interior of the outer cannula, the outer cannula defining a lateral aperture proximate the tissue piercing tip; a cutter, the cutter being movable relative to the lateral aperture of the needle to sever tissue; and a fluid source, the fluid source being configured to communicate with a portion of the needle to deploy a biopsy site marker through the marking lumen.

### Example 17

The apparatus of Example 16, further comprising a marker plug, the marker plug having a sealing portion, the sealing portion being configured to engage a portion of the needle to fluidly seal the marking lumen.

### Example 18

The apparatus of Example 16, further comprising the biopsy site marker and a marker plug, the biopsy site marker including a non-absorbable marker element and a bioabsorbable carrier, the marker element being at least partially disposed within the carrier, the marker plug being configured to be driven within a portion of the needle by fluid from the fluid source, the marker plug being further configured to drive movement of the biopsy site marker within the portion of the needle.

### Example 19

The apparatus of Example 16, further comprising the biopsy site marker, the biopsy site marker including a non-absorbable marker element and a bioabsorbable carrier, the marker element being at least partially disposed within the carrier, the biopsy site marker being pre-positioned within a portion of the needle for deployment through the marking lumen.

### Example 20

A method for deploying a biopsy site marker in tissue, the method comprising: collecting one or more tissue samples using a needle of a biopsy device to form a biopsy site in the tissue; introducing a pressurized fluid into the needle from a proximal end of the biopsy device to advance the biopsy site marker within the needle; and deploying the biopsy site marker through a marker lumen defined by a distal tip of the needle using the pressurized fluid, the act of deploying the biopsy site marker including depositing the biopsy site marker at the biopsy site.

### Example 21

The method of Example 20, further comprising sealing a portion of the needle after the step of deploying the biopsy site marker.

### Example 22

The method of any of Examples 20 or 21, further comprising detecting a pressure associated with the pressurized fluid, and diverting the pressurized fluid from the needle when the pressure exceeds a predetermined threshold.

### Example 23

The method of any of Examples 20 through 22, the step of introducing the pressurized fluid into the needle being performed after the step of collecting one or more tissue samples.

### Example 24

The method of any of Examples 20 through 23, the needle remaining in a substantially similar position relative to the tissue during the steps of collecting the one or more tissue samples, introducing a pressurized fluid into the needle, and deploying the biopsy site marker.

### Example 25

The method of any of Examples 20 through 24, further comprising coupling a marker delivery device to a tissue sample holder of the biopsy device, the step of introducing the pressurized fluid into the needle including communicating the pressurized fluid through the tissue sample holder from the marker delivery device.

### VI. CONCLUSION

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

1. A system for marking a biopsy site, the system comprising:
(a) a biopsy site marker, the biopsy site marker including a non-absorbable marker element and a bioabsorbable carrier, the marker element being at least partially disposed within the carrier;
(b) a needle, the needle including a marker lumen sized to receive the biopsy site marker;
(c) a fluid source, the fluid source being configured to communicate a pressurized fluid to the needle to advance the biopsy site marker through a portion of the needle and into the marker lumen.

2. The system of claim 1, the needle including a tissue piercing tip, the marker lumen extending through a portion of the tissue piercing tip.

3. The system of claim 1, further comprising a cutter, the needle including a tissue piercing tip, an outer cannula, and a lateral aperture, the cutter being configured to move relative to the lateral aperture to sever one or more tissue samples, the marker lumen extending through a portion of the tissue piercing tip, the marker lumen being in communication with the cutter.

4. The system of claim 1, further comprising a cutter, the needle including a tissue piercing tip, an outer cannula, and a lateral aperture, the outer cannula defining a cutter lumen and a lateral lumen, the cutter being configured to move relative to the lateral aperture within the cutter lumen to sever one or more tissue samples, the marker lumen extending through a portion of the tissue piercing tip, the marker lumen being in communication with a portion of the lateral lumen.

5. The system of claim 1, further comprising a cutter, the needle including a tissue piercing tip, an outer cannula, and a lateral aperture, the outer cannula defining a cutter lumen, a lateral lumen, and a marker transport lumen, the cutter being configured to move relative to the lateral aperture within the cutter lumen to sever one or more tissue samples, the marker lumen extending through a portion of the tissue piercing tip, the marker lumen being in communication with a portion of the marker transport lumen.

6. The system of claim 5, the marker transport lumen being fluidly isolated from the cutter lumen and the lateral lumen.

7. The system of claim 5, the marker transport lumen being disposed adjacent to the lateral lumen.

8. The system of claim 1, further comprising a marker delivery device, the fluid source being in communication with the marker delivery device, the marker delivery device being configured to releasably receive the biopsy site marker.

9. The system of claim 1, further comprising a maker delivery device, the fluid source being in communication with the marker delivery device, the marker delivery device including a cannula and a hub, the cannula being configured to receive the biopsy site marker, the cannula being further configured to communicate with the needle to transport the biopsy site marker from a portion of the cannula to the needle.

10. The system of claim 8, the marker delivery device including a sensor, the sensor being configured to detect a fluid pressure within a portion of the marker delivery device.

11. The system of claim 8, the marker delivery device including a release mechanism in communication with an interior of the marker delivery device, the release mechanism being configured to release fluid in response to contact with a fluid pressure above a predetermined threshold.

12. The system of claim 1, further comprising a tissue sample holder, the tissue sample holder being in communication with a portion of the needle, the tissue sample holder having an access port configured to permit selective communication between the fluid source and the needle via the tissue sample holder.

13. The system of claim 12, the access port of the tissue sample holder being configured to provide a substantially linear path through the tissue sample holder to the needle.

14. The system of claim 1, further comprising an access tube, the access tube being in communication with one or more portions of the needle, the access tube being configured to permit communication of fluid from the fluid source to the needle.

15. The system of claim 1, further comprising an access port, the access port being configured to provide direct access to an interior of the needle for fluid communication from the fluid source.

16. An apparatus for collecting a biopsy sample and marking tissue, the apparatus comprising:
(a) a body;
(b) a needle extending distally from the body, the needle including a tissue piercing tip, an outer cannula, and a marking lumen extending through the tissue piercing tip and in communication with an interior of the outer cannula, the outer cannula defining a lateral aperture proximate the tissue piercing tip;
(c) a cutter, the cutter being movable relative to the lateral aperture of the needle to sever tissue; and
(d) a fluid source, the fluid source being configured to communicate with a portion of the needle to deploy a biopsy site marker through the marking lumen.

17. The apparatus of claim 16, further comprising a marker plug, the marker plug having a sealing portion, the sealing portion being configured to engage a portion of the needle to fluidly seal the marking lumen.

18. The apparatus of claim 16, further comprising the biopsy site marker and a marker plug, the biopsy site marker including a non-absorbable marker element and a bioabsorbable carrier, the marker element being at least partially disposed within the carrier, the marker plug being configured to be driven within a portion of the needle by fluid from the fluid source, the marker plug being further configured to drive movement of the biopsy site marker within the portion of the needle.

19. The apparatus of claim 16, further comprising the biopsy site marker, the biopsy site marker including a non-absorbable marker element and a bioabsorbable carrier, the marker element being at least partially disposed within the carrier, the biopsy site marker being pre-positioned within a portion of the needle for deployment through the marking lumen.

20. A method for deploying a biopsy site marker in tissue, the method comprising:
(a) collecting one or more tissue samples using a needle of a biopsy device to form a biopsy site in the tissue;
(b) introducing a pressurized fluid into the needle from a proximal end of the biopsy device to advance the biopsy site marker within the needle; and
(c) deploying the biopsy site marker through a marker lumen defined by a distal tip of the needle using the pressurized fluid, the act of deploying the biopsy site marker including depositing the biopsy site marker at the biopsy site.

21.-25. (canceled)
